(19) United States
(12) Patent Application Publication
(10) Pub. No.: US 2020/0046343 A1
(43) Pub. Date: Feb. 13, 2020

(54) Interlocking Shelf and Fascia Release Mechanism

(71) Applicant: The Board of Trustees of the Leland Stanfjord Junior University, Stanford, CA (US)

(72) Inventor: Eric Anthony Kramer, Los Altos Hills, CA (US)

(21) Appl. No.: 16/656,429

(22) Filed: Oct. 17, 2019

Related U.S. Application Data
(63) Continuation-in-part of application No. 16/605,010.
(60) Provisional application No. 62/486,978, filed on Apr. 19, 2017.
(60) Provisional application No. 62/747,463, filed on Oct. 18, 2018.

(51) Int. Cl. A61B 17/04

(52) U.S. Cl. CPC A61B 17/0469; A61B 2017/0464; A61B 17/0401

(57) ABSTRACT

A tissue engagement and suturing device is provided that includes a tissue capture and engagement assembly that is slidably attachable or integrated with an access sheath, a tissue support structure of the engagement assembly includes a deformable support shelf, and an echogenic location feature, the deformable support shelf provides a surface on which a tissue layer may be secured during manipulation or suturing, the support shelf may be deformed to lay flush with the access sheath during transit into and out of a body lumen, the echogenic location feature is configured relative to the support shelf to provide visual indication of the position of the support shelf relative to a facial layer using traditional non-invasive imaging techniques.

### CROSS-REFERENCE TO RELATED APPLICATIONS

This application claims priority from U.S. Provisional Patent Application 62/747,463 filed Oct. 18, 2018, which is incorporated herein by reference. This application is a continuation-in-part of U.S. patent application Ser. No. 16/605,010 filed Oct. 13, 2019, which is incorporated herein by reference.

U.S. patent application Ser. No. 16/605,010 is a 371 of PCT application PCT/US2018/028296 filed Apr. 19, 2018. PCT application PCT/US2018/028296 claims the benefit of U.S. Provisional application 62/486,978 filed Apr. 19, 2017.

### FIELD OF THE INVENTION

The invention relates generally to a suturing device. More specifically, the invention relates to a device and method for suturing a fascial connective tissue layer that is proximal to a region under treatment.

### BACKGROUND OF THE INVENTION

Closure of large-bore access sites in the femoral artery following endovascular procedures such as Transcatheter Aortic Valve Replacement (TAVR) and Endovascular Aneurysm Repair (EVAR) is a decades-old challenge with limited solutions outside of invasive, open surgical repair of the artery. In addition to TAVR and EVAR, other surgical procedures are being adapted from manual/open procedures to a minimally invasive endovascular approach. In order to perform many of these procedures, a large-bore sheath is used to access the vascular system of a patient through the femoral artery. This sheath is placed over a dilator and inserted through the wall of a blood vessel in order to obtain this access, and may thereafter be used for guiding medical instruments such as catheters, guide wires and the endovascular devices into the circulatory system of a living being.

To perform these procedures, access into the body through layers of tissue is required to reach the femoral artery. In gaining access to different portions of the body, different layers of tissue must be separated or penetrated. These layers can include skin, subcutaneous fat, fascia (e.g., connective tissue), muscle, arteries, veins, intestine, and other organs. Often, after access through these layers is achieved and the procedure is completed, these tissues must be approximated and sealed. The function of reapproximation can vary to include hemostasis, prevent hernia, contain gastrointestinal contents, etc.

Known in the art is the “Fascia Suture Technique” that uses a suture to reapproximate the fascial layers in the roof of the femoral triangle, which may include the fascia lata, cribriform fascia, and femoral sheath with the purpose of achieving hemostasis after arteriotomy is made in the femoral artery. This technique is employed by dissecting the dermal and adipose layers of tissue along the femoral access sheath, which remains in place following the procedure. Dissection is performed both proximal and distal to the femoral sheath until the fascia layers are exposed and visually identified. A suture is passed through the fascial layers surrounding the access site, and knotted in such a way as to create a purse-string closure surrounding the femoral access sheath. As the tapered sheath is withdrawn, the knot is tightened to achieve hemostasis by closing the fascia thus containing femoral blood loss to the space bounded by the tissues that define the femoral triangle (e.g. inguinal ligament, sartorius muscle, adductor longus muscle, cribriform fascia, fascia lata, psoas major muscle, iliacus muscle, and pectineus muscle) and by resisting systolic blood pressure within the enclosed space. The wire is left in place until hemostasis is confirmed, the wire is then removed and the knot is subsequently tightened and locked. What is needed is a fascia suture technique for enabling a percutaneous, device-based solution that precludes the requirement for manual dissection and visualization of the fascial layers during closure.

### SUMMARY OF THE INVENTION

To address the needs in the art, a suturing device is provided to close a defect in the femoral artery or overlying fascia lata region of the femoral triangle; the defect may be created during percutaneous access to the femoral artery for trans-catheter interventions in the vascular system. The mechanisms described in this disclosure pertain to the identification and isolation of target tissue, the fascia lata and/or cribriform fascia, such that the defect can be closed by the passage of a suture around the circumference of the defect in the fascia lata and/or cribriform fascia. The device will enable safe and reliable closure of defects and ensure that large-bore interventions can be effectively deployed without fear of bleeding complications following the case. By offering a closure solution that is agnostic to the anatomical tortuosity of vessels and progression of disease in aging vessels, the current invention facilitates and enables large-bore trans-catheter procedures to remain minimally invasive, by avoiding the requirement for the invasive surgical closure of tortuous and diseased vessels, or of vascular access sites that are otherwise incompatible with current percutaneous closure techniques.

The current invention provides a device that includes a suture positioning assembly that is slidably attachable to an exterior of a sheath or integrated with a sheath, where a housing of the suture positioning assembly includes a housing nose, a primary suture having primary suture connectors, an end effector, and an end effector actuator, where a distal end of the housing nose includes an intravascular tip, a slide limiter, a fascia receiving surface, and a fascial isolation and support mechanism, where the housing or housing nose includes fiducial markers for visible location and positioning of the housing relative to the fascia and underlying artery, where the intravascular tip includes a blood contact indicator to receive blood flow from an artery, where the intravascular tip is configured for insertion into the artery when disposed beneath a fascial layer, where the fascia receiving surface is configured for accepting a perimeter edge of a dilated hole of the fascial layer, where the housing nose further includes an intermediary suture having intermediary suture connectors, where the primary suture connecters are configured for connection to the intermediary suture connectors, where the end effector actuator is disposed to operate the end effector to hold the facial layer hole in a position for suturing by the intermediary suture connected to the primary suture.

In one aspect of the invention, the end effector can include a straight needle, or a curved needle.

In a further aspect of the invention, a curved needle in the end effector may be configured with an attached arm connecting the curved needle to a central axis of rotation, allowing the needle to rotate in a constrained path defined by the curvature of the needle.

In yet another aspect of the invention, a curved needle in the end effector may be configured in a helical shape, allowing the needle to rotate in a path that is longer is distance that that defined by a needle rotating within a single plane.

In another aspect of the invention, the housing nose further includes a tissue capturing device that is configured to deploy from the housing nose into, beneath, or above the fascial layer and draw the dilated hole in the fascial layer to the fascia receiving surface. The tissue capturing device may include a fascial isolation and support mechanism, where the fascia isolation and support mechanism is configured for deployment to create space between the fascia and a neighboring body lumen or tissue layer. Here, the body lumen can include an artery, a vein, or a bowel, and the tissue layer can include a fascia, connective tissue, loose adipose tissue, or skin.

In a further aspect of the invention, the housing nose further includes a dilation controller, where the dilation controller is configured to change in size to alter a diameter of the dilated hole in the facial layer, where the dilation controller is configured to create resistance to the suture positioning assembly in being retracted from the facial layer along the lumen access sheath, where the created resistance is sufficient to indicate that the housing nose is securely held in place by circumferential tension of the dilated hole of the fascial layer on the fascia receiving surface. Here, the dilation control element can include an inflatable membrane, an expandable umbrella, a compliant metallic element comprised of metallic wire, interlocking metallic members, interlocking members of a rigid polymer, or a shape changing collar.

In yet another aspect of the invention, the slide limiter is configured to induce an insertion resistance along the access sheath when the housing nose is desirably positioned on the facial layer, where the desirably positioned slide limiter establishes an upper bound against which the fascia will be stabilized and prevented from creeping further up the housing nose, where the desirably positioned slide limiter prevents the housing nose from passing through the fascia beyond the fascia receiving surface.

According to one aspect of the invention, the assembly slide limiter is a material that includes an elastomer, a complaint polymer, a complaint metallic element, or an assembly of rigid elements designed to change shape upon contact with fascial layer.

In another aspect of the invention, the blood contact indicator includes a capillary, where the capillary spans from the housing nose to a proximal end of the housing, where a distal end of the capillary at the housing nose is configured to enter the body lumen, where blood flow through the capillary, or blood pressure in the capillary, or a presence of blood in the capillary is an indicator of the sheath being desirably positioned in an artery.

In a further aspect of the invention, the slidable attachment of the suture positioning assembly on the sheath includes clamping, hinged clamping, snap-fitting, stitching, or coaxial mating.

In on embodiment, the invention includes a suturing device that is slidably attachable to a sheath device, where the slidable attachment of the suturing device is configured to position a suture in an approximated state on a defect in a body lumen for suturing. Here, the body lumen can include an artery, a vein, or a bowel.

In yet another embodiment, the suturing device may be integrated with an access sheath, such that insertion of the access sheath for delivery of an intravascular device may coincide with delivery and desirable positioning of the tissue suturing device relative to the defect in the defect in a body lumen or fascia tissue layer.

Here, the body lumen can include an artery, a vein, or a bowel.

In one aspect of the invention, the suture housing or housing nose may incorporate fiducial marking features, or contrast-enhancing elements, for use in visibly positioning the suture housing in relation to the desired tissue to be manipulated. Visible identification of fiducial markers may be assisted by imaging modalities such as ultrasound, computed tomography imaging, radiographic imaging, or fluoroscopic imaging.

### BRIEF DESCRIPTION OF THE DRAWINGS

FIGS. 1A-1C show different views of a suture positioning assembly, according to embodiment of the current invention.

FIGS. 2A-2C show the intravascular tip inserted to an artery when disposed beneath a fascial layer, according to the current invention.

FIGS. 3A-3G show an exemplary detailed procedure of the device operation of the internal structures of the device, according to the current invention.

FIGS. 4A-4C show embodiments of the shapes of example end effector shapes, according to the current invention.

FIGS. 5A-5C show different views of an alternate suture positioning assembly, according to embodiment of the current invention.

FIGS. 6A-6E show alternative mating mechanisms of the suture positioning assembly with the sheath, according to the current invention.

FIGS. 7A-7C show different embodiments of the housing nose having a dilation controller that is configured to change in size to alter a diameter of said dilated hole in facial layer, according to the current invention.

FIG. 8 shows a flow diagram of the implementation and use of the current invention.

FIGS. 9A-9E show an embodiment of the suture positioning assembly comprised of interlocking but independently sliding members so as to control the dilation of a fascial defect in a fascial layer so as to remove the fascial layer from the fascia receiving surface following manipulation and passing of sutures to close the fascial defect.

FIGS. 10A-10E show several embodiments of a tissue capturing device, deployable distal to the fascia layer so as form mechanical support distal to the fascial layer and allow it to be drawn up and into the fascia receiving surface, while providing geometry to allow for the unimpeded passage of end effectors during puncturing of the fascia for the passing of sutures in order to close a defect in the fascia layer.

FIGS. 11A-11G show the use of imaging techniques to reflect a signal from an imaging probe off of a locating feature designed to accommodate and receive a fascia layer for manipulation and suturing. Several embodiments of a locating feature that is visible using current imaging modalities are presented.

FIGS. 12A-12C show an embodiment of an end effector wherein the travel of the end effector is constrained by mechanical mating with a rotating axis, such that the end effector may reliably reach its target to form a barb/cuff complex for subsequent threading through a fascia layer and resultant suture passing.

FIGS. 13A-13C show an embodiment of end effectors in a helical configuration so as to offset the destination of the end effectors from the starting plane, whereby the offset permits a greater degree of rotation of end effectors at their proximal end without interference with their target along a semicircular path, the cuffs that are used to form a barb/cuff complex during suture passing and subsequent closure of a fascial defect.

### DETAILED DESCRIPTION

The current invention provides a surgical procedure and closure device that approximates and closes a defect in a tissue layer, such as a tissue layer superior to a blood vessel. In one example, the tissue defect may arise from the external introduction of an instrument for vascular or otherwise internal access to the body, e.g. percutaneous or surgical intervention. The tissue layer may be the femoral sheath, fascia lata and/or cribriform fascia that covers the roof of the femoral triangle, or other connective tissue layer e.g. abdominal fascia. The closure of such a tissue may allow for hemostasis to occur in the enclosed femoral triangle in order to create a natural method of tamponade around the femoral artery after removal of the procedural sheath. The ability to exert a greater compressive force around the arteriotomy from closure of the fascia above and the surrounding borders of the femoral triangle may allow the arteriotomy to naturally resolve. The anatomy of the femoral triangle includes the floor made up of the following muscles from lateral to medial: iliacus, psoas major, pectineus, and adductor longus; the superior border is made up of the inguinal ligament; the medial border is the adductor longus muscle; and the lateral border is comprised of the sartorius muscle. The current invention eliminates procedural steps that include opening the skin, manually dissecting down to the fascia, and directly suturing the fascia to close these internal tissues.

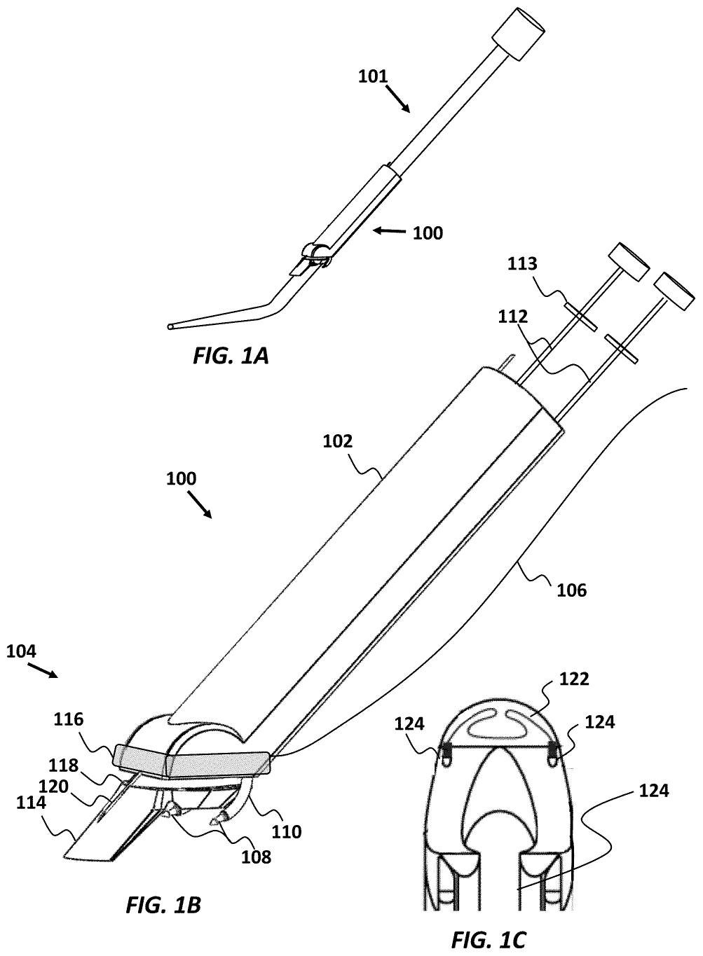

Turning now to the drawings, FIGS. 1A-1C show different views of a suture positioning assembly 100 that is slidably attachable to an exterior of a sheath 101. As shown in FIGS. 1B-1C, the housing 102 of the suture positioning assembly 100 includes a housing nose 104, a primary suture 106 having primary suture connectors 108, end effector 110 that can be independently deployed, and an end effector actuator 112, wherein a distal end of said housing nose comprises an intravascular tip 114, a slide limiter 116, and a fascia receiving surface 118, where the intravascular tip 114 comprises a capillary tube 120 to receive blood flow from an artery.

In one aspect of the invention, the assembly slide limiter includes a material that includes a compliant elastomer that, upon contact with the proximal surface of the fascia, will deform and/or buckle, adding additional cross-sectional area to the upper bounding surface of the slide limiter, creating a broader upper bound of the slide limiter with which to prevent the fascia from creeping up further up the housing nose beyond the fascia receiving surface. Here, the slide limiter can be the elastomer, complaint polymer, complaint metallic element comprising nitinol, or an assembly of rigid elements designed to change shape upon contact with fascial layer.

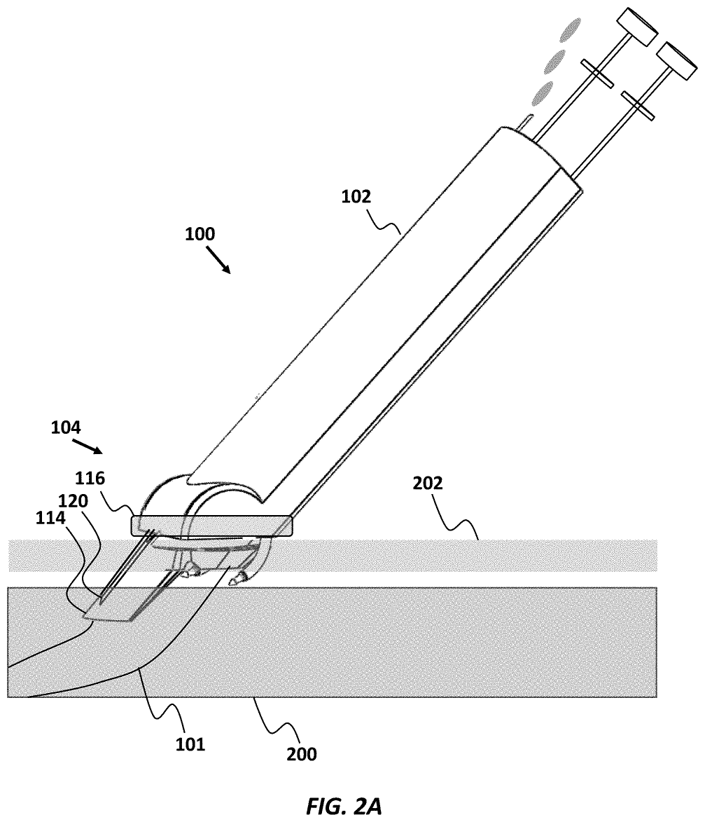

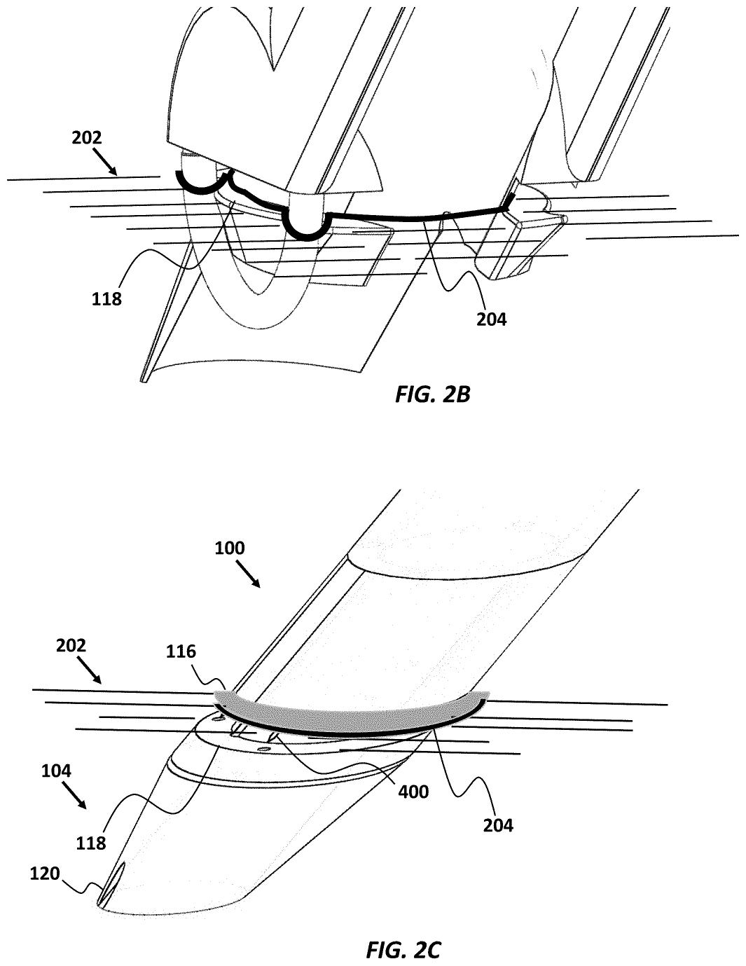

FIG. 2A, shows the intravascular tip 114 inserted to artery 200 when disposed beneath a fascial layer 202, where the fascia receiving surface 118 (for example having a ledge-shape) is configured for accepting a perimeter edge of a dilated hole (defect) 204 of the fascial layer as shown in FIGS. 2B-2C, where in FIG. 2B the slide limiter 116 is not shown for clarity. Further shown in FIG. 2C is an alternate embodiment of the suture positioning assembly 100 that has a conical housing nose 104, and linear elements for the end effectors 400 and suture assemblies described below.

FIG. 1C shows the housing nose 104 further including an intermediary suture 122 having intermediary suture connectors 124, where the primary suture connecters 108 are configured for connection to the intermediary suture connectors 124. According to the invention, the end effector actuator 112 is disposed to operate the end effector 110 to hold the facial layer hole in a position for suturing by the intermediary suture connected 112 to said primary suture 106.

The current invention enables one to approximate tissues during or following interventional medical procedures, such as open surgery, laparoscopic surgery, endoscopy, endovascular surgery and procedures, and cardiac catheterizations is portrayed. The invention may be used in a percutaneous or open surgical fashion and approximate a variety of tissues, such as skin, subcutaneous fat, fascia, muscle, or vascular tissue. Further, the tissue approximation device may be used with the intent of sealing tissue to achieve vascular hemostasis, prevent hernias, obtain watertight or airtight closure of tissue layers, or approximate and close disjointed tissues, for example. Tissue sealing of any of the previously described tissues may be achieved by a variety of mechanical techniques such as suture-like materials, clips, hook-and-loop fasteners, patches, plugs, clamps, and/or biological and synthetic adhesives. The invention may be placed directly on the tissue of interest or be integrated with an instrument of preference, such as a cardiovascular sheath, a guidewire for maintaining access to a vessel or procedural space, or a laparoscopic surgery port, trocar, or other surgical or procedural access instrument.

FIG. 1A shows an example interface of the suture positioning assembly 100 as coaxially mated with a tubular access instrument 101, in this case, a cardiovascular access sheath. The cardiovascular sheath 101 serves as a guide along which the device 100 travels during operation. The coaxial mate enables the device 100 to follow the path of the access instrument 101 through the skin, and any subcutaneous adipose or loose connective tissues, in order to position itself against an access-induced defect in a known anatomical structure, e.g. the cribriform fascia, fascia lata, or femoral sheath. The device 100 may be used to manipulate a known anatomical structure in order to approximate and close an access-induced defect in the structure. The device is inserted, manipulated, and subsequently removed to induce the desired tissue closure effect, leaving behind only the mechanical components necessary to maintain tissue closure.

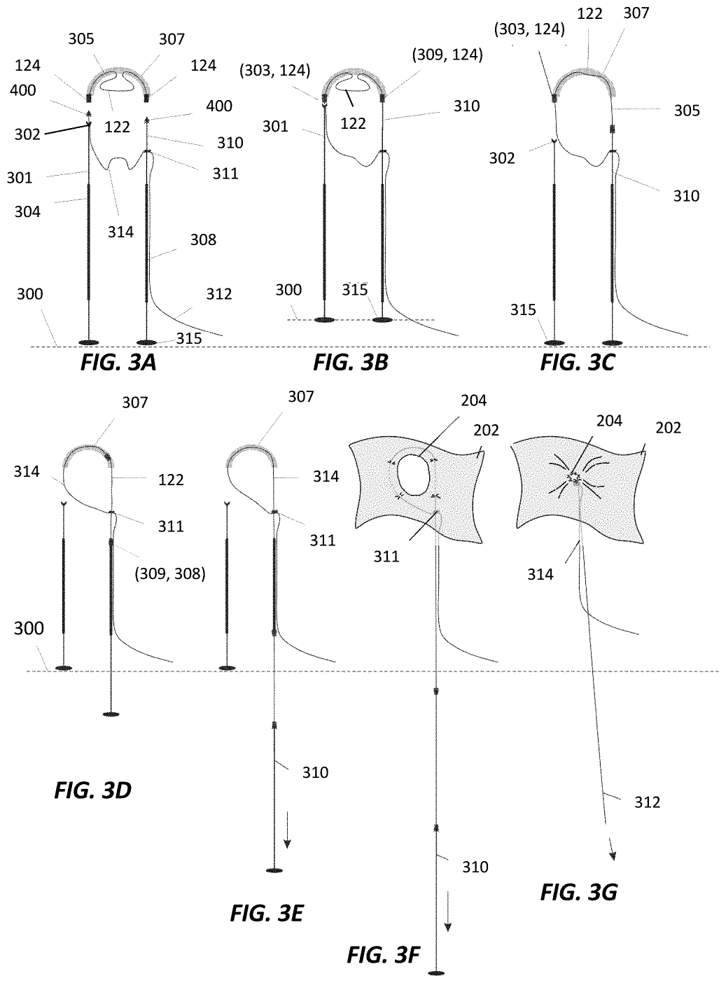

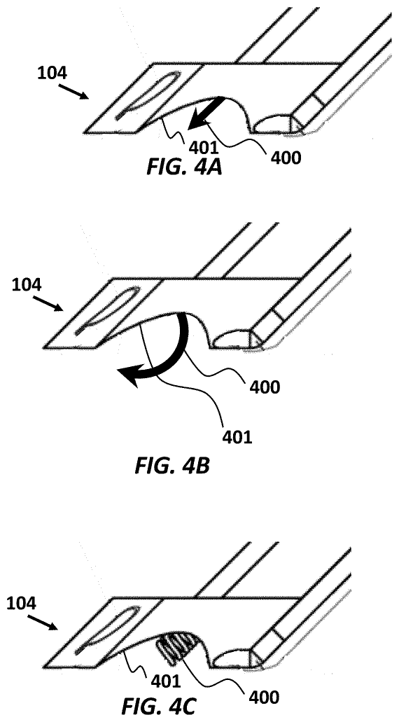

FIGS. 3A-3G show an exemplary detailed procedure of the device operation of the internal structures of the device 100 and their designated operations to deliver suture-mediated closure to a tissue defect in a subcutaneous tissue layer of interest. In this example, once the user has passed the streamlined or tapered device nose 104 around the access instrument 101 and through the superficial tissue layers to reach the level of the subcutaneous tissue of interest 200, the user may deploy end effectors 400 (see FIGS. 4A-4C) to capture the tissue of interest 202 (e.g., fascia) and the retract the tissue of interest 202 into the receiving feature of the device nose 104 effecting the desired fold or “tent” in the tissue of interest. The device is now primed for suture deployment. FIGS. 3A-3G show a schematic views of the internal structures and mechanisms of the device 100 as stored for transportation and handling. Reference position 300 indicates the stored position of end effectors 400. End effector 400 may be a barbed suture needle that is mechanically mated with its axial shaft 301 via a press-fit or similarly tenuous junction 302 capable of releasing end effector 400 from its axial shaft 301 upon the application of tension to the three-part system that resides and travels within device body lumen 200. One end 314 of a suture system (314, 311, 312), which in combination make up the primary suture 106, may be permanently affixed to end effector 303 such that any movement of end effector 303 is followed by its attached suture end 314. As shown previously, FIG. 3A details intermediary suture 122 as attached on both ends to suture cuffs 124. The stored portion of intermediary suture 122 may reside in the nose 104 of the device body 100, where upon the application of tension to one end of the intermediary suture 122, the entirety of intermediary suture 122 will travel within and follow the curved path of an interior feature 307 of device nose 104. The interior feature 307 may be a shelf or channel that provides a radial force to the intermediary suture and subsequent flexible sutures as they are drawn through the feature around the cylindrical access instrument, so as to prevent contact of the suture with the device body. The interior feature 307 may not restrict any suture from dropping out of the “active surface” of device body 100.

End effector 400, which may be a barbed needle tip, is permanently affixed to its axial shaft 310 housed within device body lumen 304. The axial shaft 310 of the end effector 400, as described previously, is stored such that it passes through a pre-tied surgical knot 311 comprised of the junction between a suture end 314 that is stored internal to the device within device lumen 304 and an opposing suture end 312 that may be stored external to the device or within lumen 304.

When tissue has been folded or tented into the receiving surface, for example a concave feature or planar shelf, of device nose 104 via deployment and retraction of end effectors 400, the user may actuate end effectors 400 by applying compressive force to their axial shafts 301 and 310 via handles or actuators 315 to pass the end effectors through the fold in the tissue of interest, as shown in FIG. 3B. The passing of end effectors 400 creates two straight passages through folded tissue on either side of the access instrument e.g. cardiovascular sheath for a total of four holes radially arranged around the defect in the tissue of interest. As such, the end effectors enter the tissue from the side of the tissue layer that is proximal to the device user, puncture into and return out of the tissue on the side of the tissue layer that is proximal to the device user. The actuators of axial shafts 301 and 310 may now reside in reference position 300 as shown in FIG. 3B. End effector 400 may have now formed a mechanical mate with suture cuff 124 forming barb/cuff complex (400, 124), and the opposing effector 400 may have likewise mated with suture cuff 309. Stored suture end 314 may have been partially drawn out from lumen 307.

Shown in FIG. 3C, axial shafts 301 and 310 may be retracted from the tissue. Due to the suture/cuff complexes 303, 124, the axial shaft 301 and tenuous mechanical junction (e.g. socket, or cradle) 302 may be allowed to travel back through the tissue having detached from end effector 400. Actuator 315 of axial shaft 301 may return to reference position 300 and remain there. Axial shaft 310 is now mechanically coupled to barb/suture cuff complex 309, 124, stored and resident intermediary suture 305, 122, barb/suture cuff complex 303, 124 and previously stored suture end 314. This 6-part system may now be drawn in tension, as shown in FIG. 3D, by actuator handle 315 of axial shaft 310 through the pre-tied suture knot 311, which remains housed in the distal end of the axial shaft port. Actuator handle 315 may now surpass reference position 300 as the 6 part system is drawn through the tissue, and axial shaft 310 is drawn away from the device, towards the device user. By drawing this 6 part system through the structural guide channel 307 in the nose 104 of the device 100, the intermediary suture and cuff complexes draw the suture end 314 through pre-tied surgical knot 311, the remaining end of which belongs to the same filament of suture 312. The tissue defect is now surgically tied, and while, in the event of a cardiovascular access closure, the guiding wire must remain intraarterial, the access instrument may be removed by the user while the user applies tension to suture end 312, the suture “rail”, and pushes suture knot 311 towards the defect 204 in the tissue of interest 202, as shown in FIG. 3F. The intermediary suture 112 and cuff complexes may now be trimmed from the single knotted suture, and the knot may be further pushed either manually or via a user-activated tensioning mechanism within the device so as to create a purse-string closure of defect 204 using a U-stitch or analogous surgical closure pattern in the tissue of interest 202 as shown in FIG. 3G. In the case of a cardiovascular access closure, the guidewire is still in place. As the user determines the closure to be effective, the user or device may continue to apply tension to suture “rail” 312, and compressive force to knot 311 as the user removes the guiding wire and completes the knotted, purse-string closure of the tissue of interest. For the indication of achieving hemostasis after closure of the tissue of interest, the device may embed indicators in the housing, such as a cannula, needle or wick to inform the user if hemostasis was or was not achieved after securing the approximated tissue.

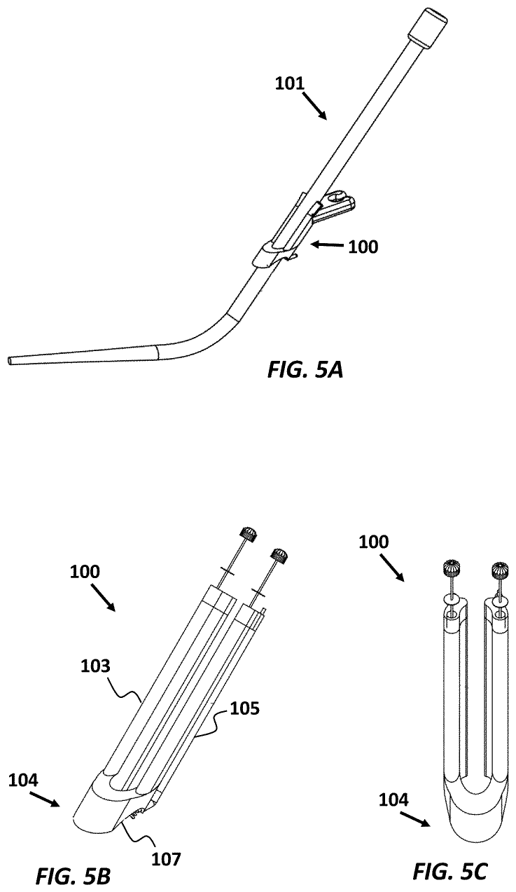

To achieve a coaxial mate with the suture positioning assembly 100 as shown in FIG. 1A, the tissue approximation device may be snapped, pressed, or otherwise mechanically secured onto the instrument, e.g. a cardiovascular access sheath 101 via an axial slit 124 (see FIG. 1C, FIGS. 5A-5C). The axial slit 124 and corresponding structural boundaries may be, but are not limited to a blunt, sharp, circular, convex, or cone shape to allow tissue, such as subcutaneous fat, to slide over the tissue approximation device and mitigate damage, entanglement or interference with the tissue before reaching the desired tissue layer and tissue defect to be approximated, which may include a subcutaneous tissue layer such as the fascia or arterial wall.

In a further aspect of the invention, the slidable attachment of the suture positioning assembly on the sheath to achieve coaxial mating of the suture positioning assembly with the sheath includes clamping, hinged clamping, clamping via hinged cams, springed hinges, snap-fitting, stitching, tongue-and-groove mating, dove-tail mating, ratchet mating, threaded connnectors or coaxial mating. The suture positioning assembly may be a single deformable component so as to achieve the coaxial mate with the sheath, or it may be comprised of discrete halves to be radially/circumferentially oriented around the sheath so as to mate with each other and create a coaxial mate, or a mate with which the central axes of the suture positioning assembly and of the sheath are offset but remain parallel.

Shown in FIGS. 5A-5C is one possible embodiment of a suture positioning assembly 100 to achieve vascular hemostasis. FIG. 5A shows one embodiment of the invention, where a device is provided as an integrated sheath and closure device. In further embodiments, the device may be equipped with a streamlined or tapered introductory access nose 104 intended to dilate any tissue (e.g. skin and adipose tissues) that surrounds the access sheath while traveling along the sheath towards the tissue defect of interest. To appropriately approximate to and manipulate the tissue of interest, the tissue-facing surface, or “active surface” of the device nose 104 may be angled relative to the axis of the device body 100 so as to achieve a parallel approximation with the tissue layer of interest. The “active surface” of the device may thus be oriented relative to the anterior longitudinal surface of the device body 100 at an angle of between 14 and 61 degrees. The active surface of the device may also follow convex contours designed to mate seamlessly with the tissue layer of interest, around the defect of interest. The device is equipped with two symmetrical, axial lumens 103 through which tissue end-effectors may pass and be articulated, directed, or manipulated. The device is equipped with two additional symmetrical lumens 105 that likewise house tissue end effectors that may be passed, articulated, directed or manipulated to puncture the tissue of interest and deploy mechanical closure components, e.g. sutures. Lumens 105 may contain baffles interior to the lumen in order to guide any flexible internal tissue end effectors in the appropriate direction. The device is equipped with a concave feature 107 within the nose 104 that will serve as a contour along which to shape the tissue and tissue defect, as well as providing a dedicated volume of space within which the tissue may be manipulated during the closure operation. In no way is the dedicated volume created by concave feature 107 restrictive of the area in which end effectors may pass or within which tissue may be manipulated.

The material of the device, particularly in, but not limited to the location of the nose 104 may be sufficiently flexible so as to allow the elastic deformation of the nose or device body and subsequent coaxial mating of the suture positioning assembly 100 with the access sheath 101. The mating surfaces of the suture positioning assembly 100 with sheath 101 may incorporate gaskets, bushings, springs, hydrophilic coatings or other mechanism(s) to ensure a tight, coaxial sliding junction with the access instrument. The independence of the suture positioning assembly 100 from the sheath 101 enables the device to perform the intended tissue manipulation either at the start of a procedure or at the end of a procedure, without the mandate that the access sheath be removed and “threaded” through the device in order to obtain a coaxial orientation of the two components.

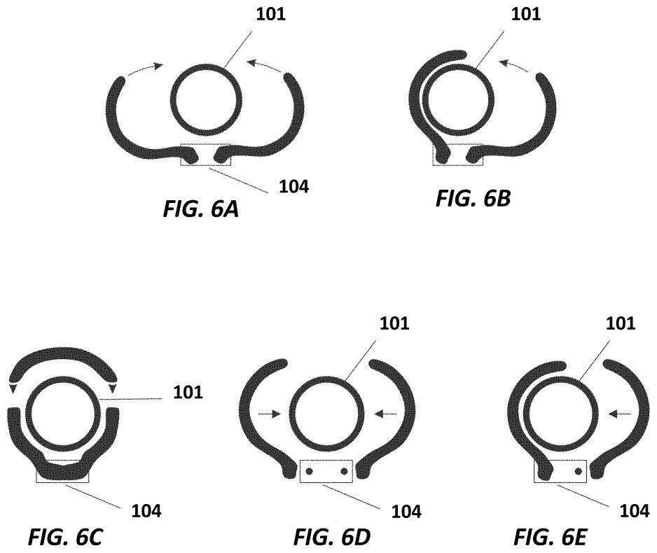

FIGS. 6A-6E show alternative mating mechanisms of the suture positioning assembly 100 with sheath 101, e.g. cardiovascular sheath. The device 100 may coaxially mate to a cylindrical instrument 101 via a single hinge (FIG. 6A) or dual-hinge (FIG. 6B) clamshell-type approximation mechanism, such that one or both halves of the end effector of the device may swing open, allowing the device to be placed around the sheath and subsequently closed and secured. Rather than an axial slit or clamshell, the device may also occupy a modular form factor as depicted in FIG. 6C. In one embodiment, the nose 104 or the body of the device 100 to be mated with an access instrument may be removed or disconnected from the device (FIG. 6D) to allow coaxial mating with the sheath, and subsequently replaced to form a circumferential sliding junction with the access instrument. The device body may also be split on the transverse plane as in FIG. 6D such that one half of the device is bound to the other around the access instrument, or such that each half is bound to a core body member. FIG. 6E depicts one half of the device bound to a core body member; the remaining modular half may be joined to the core body member around the access instrument.

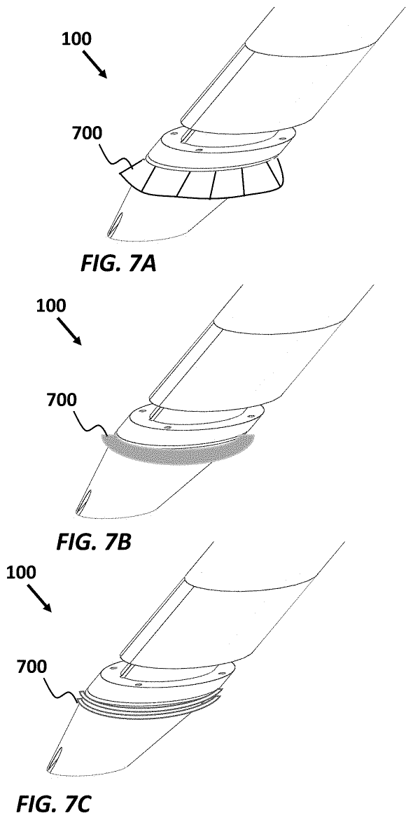

According to further embodiments of the invention, the housing nose 104 can also include a dilation controller, where the dilation controller is configured to change in size to alter a diameter of said dilated hole in facial layer 202, where the dilation controller is configured to create resistance when the suture positioning assembly 100 is retracted from the facial layer 201 along said lumen access sheath 101, where the created resistance is sufficient to indicate that the housing nose 104 is securely held in place by circumferential tension of the dilated hole 204 of said fascial layer on said fascia receiving surface. FIGS. 7A-7C show different embodiments dilation control element 700 that can include an expandable umbrella (see FIG. 7A), an inflatable membrane (see FIG. 7B), and a shape changing collar (see FIG. 7C). Here, the dilation control element can be deformable underneath the fascia layer so as to form an “anchor” or “T-tag”-style expansion device so as to prevent the user from pulling the device out of position while operating. In one embodiment, this feature could then be relaxed to reduce its cross section to original size and allow the device to be removed/slid back along the sheath towards the user thus disengaging from the fascia.

In one embodiment, the end effectors may be a coiled wire with sufficient stiffness to puncture and secure into a biological tissue of interest for subsequent pushing, pulling, and analogous tissue manipulation. The end effectors may embody or utilize similar tissue securement mechanisms such as opposing graspers, hooks, barbs, suction, or adhesives. After approximation of the suture positioning assembly 100 to the tissue of interest 202, the end effectors 400 may be secured to the tissue of interest by rotating and applying axial compression to their axial shafts via handles/actuators at the end of the end-effector axial shaft proximal to the user, in the direction of the tissue of interest, which effectively captures the tissue of interest e.g. fascia. The travel of the end effectors is limited by a mechanical hard stop, or “shoulder” feature 113 (see FIG. 1B) on the axial shaft of the end effectors. Once in contact with the device body 100, the “shoulder” 113 will stop travel of the end effectors so as to avoid damage of delicate tissues or structures beneath the tissue of interest, e.g. fascia. The tissue of interest may then be retracted into the interface surface, such as the concave feature 401 of FIGS. 4A-4C of the device nose 104 by applying tension to shaft of the end effectors (not shown0, creating a folding or “tenting” effect of the tissue of interest within the nose 104 of the device body 100.

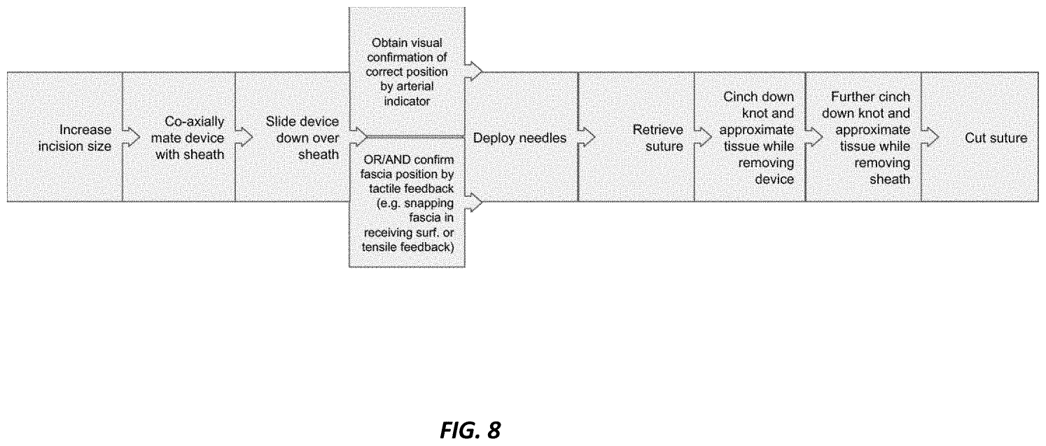

FIG. 8 shows a flow diagram of the implementation and use of the current invention that includes increasing the incision size, co-axially mating the device with a sheath, sliding the device down over the sheath, then obtaining visual confirmation of the correct positioning of the device by an arterial indicator, and/or confirming the fascia positioning by use of a tactile feedback, such as snapping fascia in receiving surface or a tensile feedback. The next steps include deploying the needles, retrieving the suture, cinching down the knot and approximating the tissue while removing the suturing device, then further cinching down the knot and approximating the tissue while removing the sheath, and finally cutting the suture.

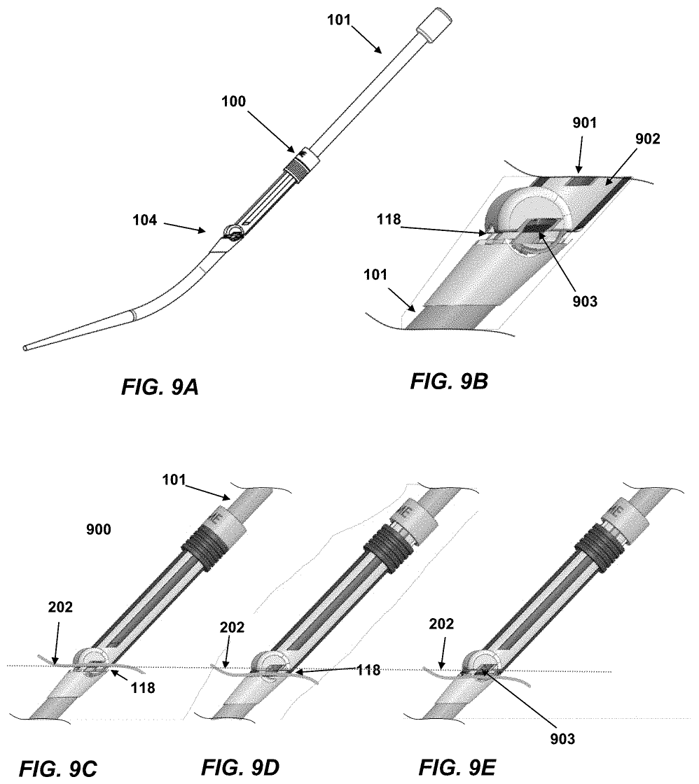

FIG. 9A shows an embodiment of a suture positioning assembly 100 that is slidably attachable to an exterior of a sheath 101. In FIG. 9B, housing nose 104 of suture positioning assembly 100 incorporates a dilation controller with radially oriented interlocking members 901 and 902, where member 901 is axially distensible relative to 902 such that a tapered dilation tip 903 on the distal end of member 901 may be advanced distal to fixed member 902, where fixed member 902 is mechanically integrated to housing nose 104. Shown in FIGS. 9C-9D, tapered dilation tip 903, when advanced by the deployment of interlocking member 901 relative to fixed interlocking member 902, may increase the dilation of fascial hole 204 in fascial layer 202 such that the fascial layer may be disengaged from the fascia receiving surface 118 until fascial layer 202 may be disengaged from housing nose 104. FIG. 9D shows dilation tip 903 advancing fascial layer 202 off of fascial receiving surface 118 via dilation of fascial hole 204. In FIG. 9E, fascial layer is sufficiently disengaged from fascia receiving surface 118 such that housing nose 104 in conjunction with suture positioning assembly 100 may be slidably withdrawn from engagement with fascia layer 202 following deployment of sutures, and suture positioning assembly 100 may be withdrawn from the body lumen or access site such that sutures may be cinched and secured for completion of suture passing and closure of fascial hole 204 in fascia layer 202.

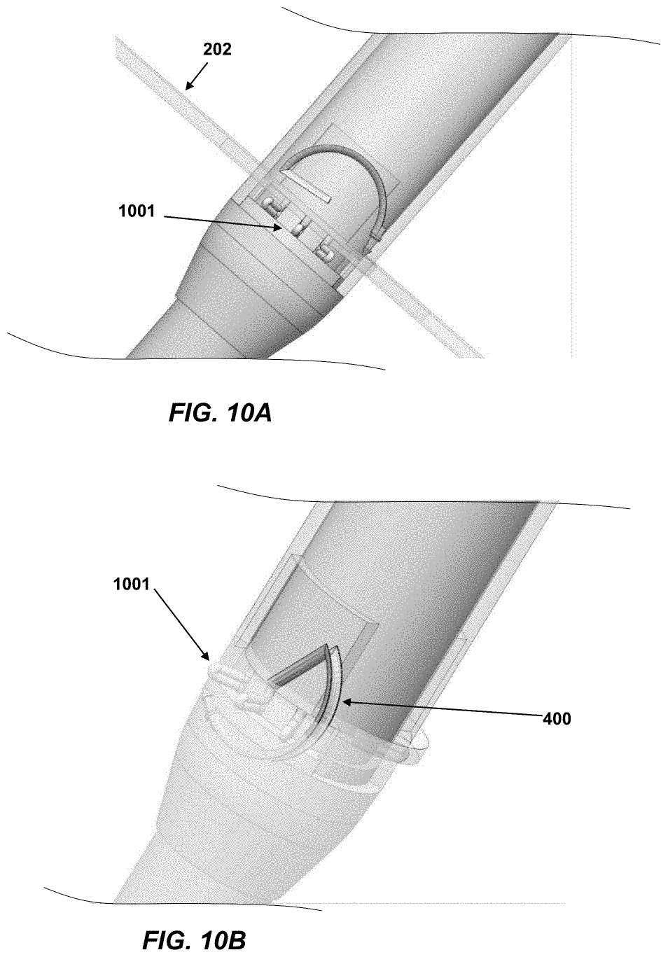

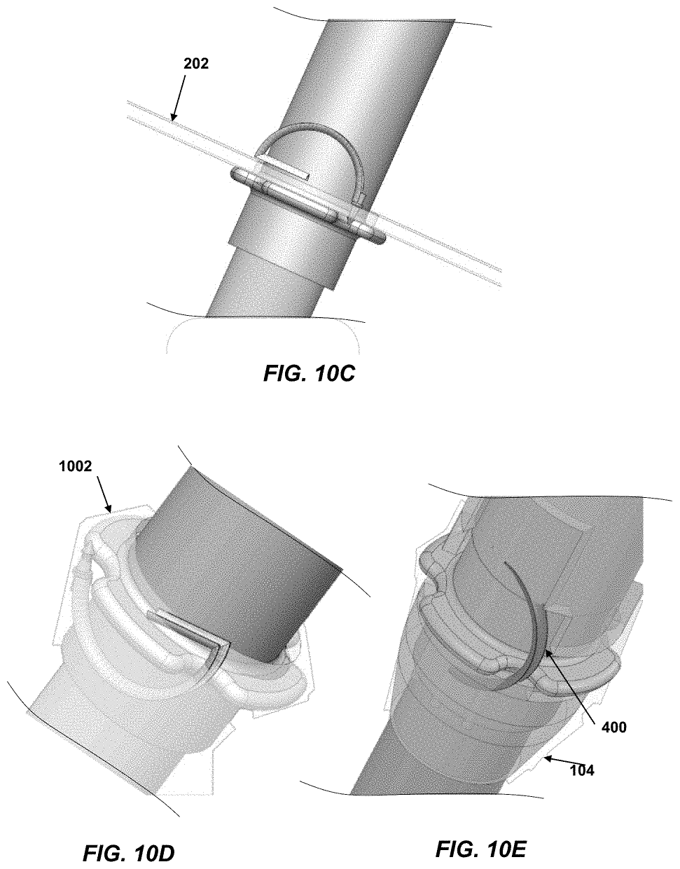

FIG. 10A depicts a tissue capturing device embodied by a circumferential array of shaped metallic wires 1001 around and housing nose 104 of suture positioning assembly 100. The array of shaped metallic wires 1001 is an embodiment of a fascia isolation and support mechanism that may be deployed from a stored configuration, where the array of shaped metallic wires 1001 may be tensioned against suture positioning assembly 100 until such time as they need to be deployed proximal or distal to fascia layer 202. Once deployed distal to fascia layer 202, the array of shaped metallic wires 1001 may be retracted by drawing housing nose 104 and thus fascial layer 202 up to the fascia receiving surface for manipulation and suturing. In FIG. 10B, the arrangement of end effectors 400 may be seen relative to the array of shaped metallic wires 1001 such that end effectors 400 may travel along their path through fascia layer 202, which is supported distally by the deployment of the array of shaped metallic wires 1001, unimpeded by the deployed array of shaped metallic wires 1001. As such, fascial layer 202 may be supported both proximally and distally during manipulation, puncturing, and suturing.

FIGS. 10C-10E depict an alternate embodiment of a tissue capturing device, wherein the tissue capturing device is comprised of a deployable array of shaped metallic wires in a braided configuration. As such, the braided array of shaped metallic wires 1002 may be deployed identically to the array of shaped metallic wires 1001 in order to provide support of fascial layer 202 during manipulation and suturing, yet the braided array of wires 1002 may provide additional circumferential support of fascial layer 202 relative to wires 1001. FIGS. 10D-10E depict the shape of a braided array of shaped metallic wires 1002 such that end effectors 400 may deploy through fascia layer 202 unimpeded by the support structure formed after deployment of the braided array of shaped metallic wires 1002 in order for end effectors 400 to complete suturing and closure of fascial defect 204 in fascial layer 202.

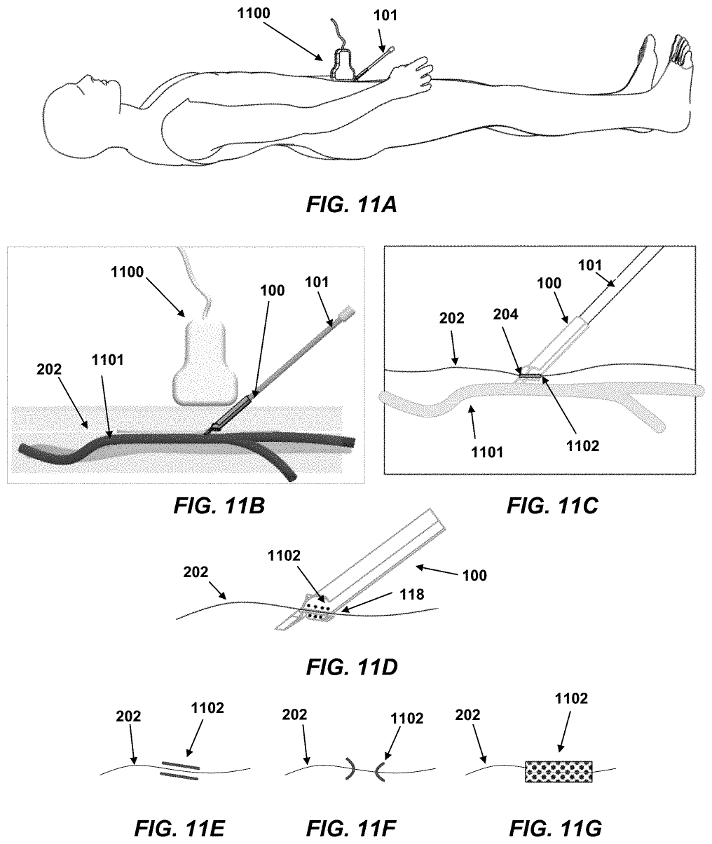

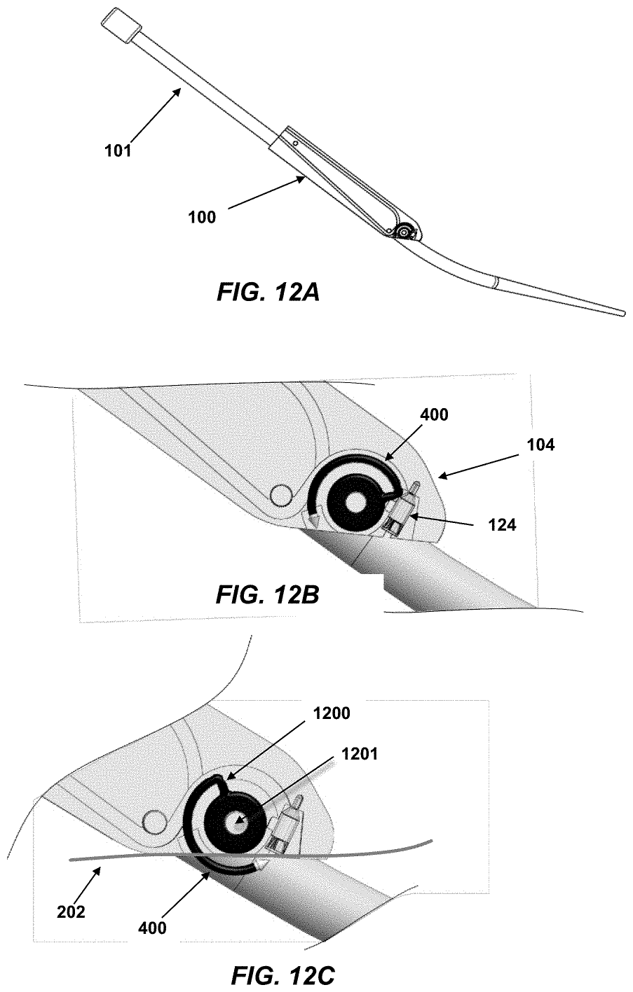

FIGS. 11A-11G show the identification, location and positioning of suture positioning assembly 100 and fascia receiving surface 118 relative to fascia layer 202. FIG. 11A indicates the positioning of an ultrasound imagery probe 1100, oriented such that the field of view of the ultrasound image is parallel and coincident with the central axis of sheath 101. FIG. 11B depicts suture positioning assembly 100 slidably engaged with sheath 101 at its entry point through fascia layer 202 and further distally into a body lumen 1101. FIG. 11C depicts the field of view under ultrasound imagery comprising multiple echogenic features that generate contrast in the ultrasound image, primarily fascia layer 202, body lumen 1101 and an echogenic locating feature 1102 that coincides with fascia receiving surface 118. By the integration of echogenic location feature 1102 with fascia receiving surface 118, the use of ultrasound imaging via probe 1100 will allow for the visual confirmation of engagement of fascia layer 202 with fascia receiving surface 118 such that deployment of end effectors 400 for the passing of a suture through the fascia layer may be performed with visual affirmation of appropriate positioning and deployment of end effectors 400 through facia layer 202. FIG. 11D depicts one embodiment of echogenic location feature 1102, wherein topographical indentations in a metallic or otherwise echogenic material along the body of suture positioning assembly 118 that is just proximal to fascia layer 202, as well as topographical indentations in a metallic or otherwise echogenic material along the body of housing nose 104 that is just distal to fascia layer 202, between which lies the fascia receiving surface 118, form a proximal and distal visual indicator of the engagement of fascia layer 202 with fascia receiving surface 118. FIG. 11E shows another embodiment of echogenic location feature 1102 comprised of linear metallic or otherwise echogenic features both proximal and distal to fascia layer 202. FIG. 11F shows yet another embodiment of echogenic location feature 1102 wherein metallic or otherwise echogenic features are arranged on suture positioning assembly 100 and housing nose 104 in a semicircular configuration within the visual plane of imagery, so as to straddle or cradle the fascia layer 202 with the visible features of the echogenic location feature 1102. Visibility of echogenic location feature 1102 in this embodiment may be achieved via texturing of a metallic or otherwise echogenic material comprising the echogenic location feature 1102. In yet another embodiment of echogenic location feature 1102, FIG. 11G depicts a texturization of the location feature 1102 in a bulk indicator that seeks to provide visual confirmation of linear alignment of echogenic location feature 1102 which coincides geometrically with fascia receiving surface 118, with fascia layer 202. Echogenicity of the location feature 1102 may again be achieved by texturization of a metallic or otherwise echogenic material comprising the feature so as to be visible in the imaging plane.

FIG. 12A shows an alternative embodiment of suture positioning assembly 101 slidably attached to sheath 100. In FIG. 12B, End effectors 400 located within housing nose 104 are shown in a semicircular geometry in this embodiment, joined by a connecting arm 1200 to a central axis of rotation 1201. The fixed geometry of end effector 400 joined by connecting arm 1200 to axis of rotation 1201 ensures that upon deployment and rotation of end effectors 400, the barbed component of end effector 400 may travel along a preset path of rotation so as to ultimately mate with cuff 124 to form suture/cuff complex 303,124 as shown in FIGS. 3B-3C. FIG. 12C depicts penetration of fascial layer 202 by end effectors 400 along their path to form the mating suture/cuff complex 303,124.

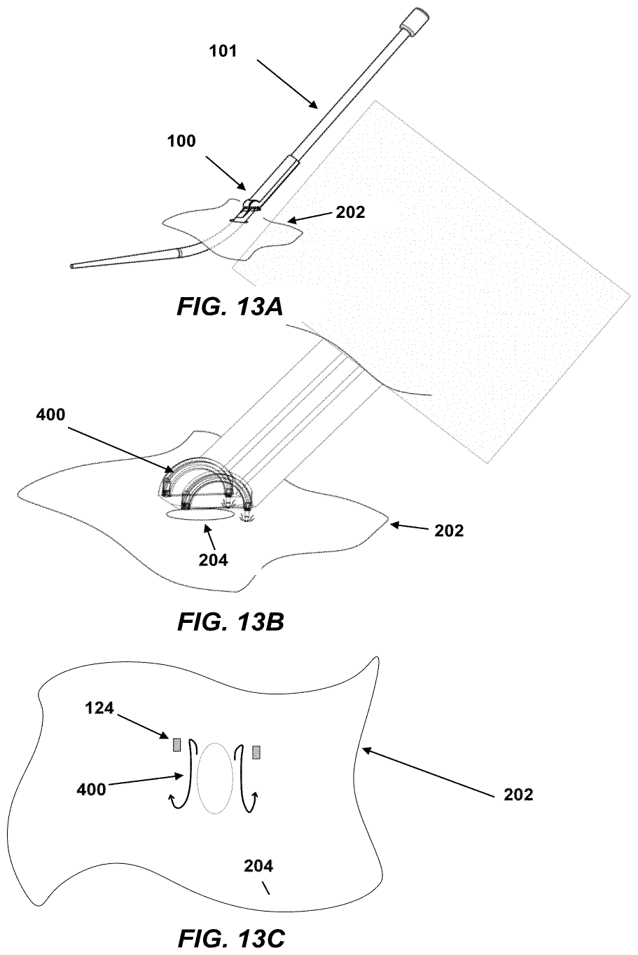

FIG. 13A shows yet another embodiment of a suture positioning assembly 100 slidably attached to sheath 101 passing through fascia layer 202. In this embodiment, end effectors 400 are shown in FIG. 13B to pass through fascia layer 202 around fascial defect 204, where FIG. 13C depicts end effectors 400 in a helical configuration, such that the deployment of end effectors 400 along a helical path allows for the formation of barb/cuff complex 303, 124 while allowing the distal end of end effector 400 to occupy the same lateral space as cuff 124, albeit with an offset. As such, end effector 400 may be configured in a longer arc length in this embodiment than that permitted in FIGS. 12A-12C, Given that the path of travel of the helical end effector in FIG. 13C does not interfere with the target destination of cuff 124 as end effector 400 travels along its path of deployment to form barb/suture complex 303, 124.

The present invention is capable of many variations in detailed implementation, which may be derived from the description contained herein by a person of ordinary skill in the art. For example, the needles themselves could be curved or straight, flexible or rigid, etc. while still performing the mating of “barb” with “cuff” by traversing the tissue layer once, twice, or up to four times per needle.

What is claimed:

1) A tissue layer engagement device, comprising:
a tissue capture and disengagement assembly that is slidably attachable or integrated with the exterior of a sheath, wherein a tissue support structure of the tissue capture and disengagement assembly comprises a static or deformable support shelf, wherein the support shelf is configured to deform itself to enable or limit travel of the tissue support structure relative to a tissue layer along the tract of an access conduit into a body lumen, wherein the tissue support structure further comprises an echogenic location feature with a contrast enhancing geometry to be visible when using traditional non-invasive imaging techniques, wherein the echogenic location feature is used to visually confirm the position of the tissue support structure relative to a tissue layer to be engaged along the tract of an access conduit through the skin into the body lumen.
